(19) United States
(12) Patent Application Publication
(10) Pub. No.: US 2025/0276108 A1
(43) Pub. Date: Sep. 4, 2025

(54) REVERSIBLY STICKING METALS AND GRAPHITE TO HYDROGELS AND TISSUES

(71) Applicant: University of Maryland, College Park, College Park, MD (US)

(72) Inventors: Srinivasa R. Raghavan, Columbia, MD (US); Wenhao Xu, College Park, MD (US); Irene Orueta-Ortega, College Park, MD (US); Faraz A. Burni, Silver Spring, MD (US); Amir H. Mohammadi, Hyattsville, MD (US)

(21) Appl. No.: 19/067,488

(22) Filed: Feb. 28, 2025

Related U.S. Application Data
(60) Provisional application No. 63/559,821, filed on Feb. 29, 2024.

(51) Int. Cl. A61L 27/06; A61L 27/04; A61L 27/08; A61L 27/16; A61L 27/52

(52) U.S. Cl. CPC A61L 27/06; A61L 27/047; A61L 27/08; A61L 27/16; A61L 27/52

(57) ABSTRACT

Hard, electrical conductors (e.g., metals or graphite) can be adhered to soft, aqueous materials (e.g., hydrogels, fruit or animal tissue) without the use of an adhesive. The adhesion is induced by a low DC electric field. As an example, when 5V DC is applied to graphite slabs spanning a tall cylindrical gel of acrylamide (AAm), a strong adhesion develops between the anode (+) and the gel in about three minutes. This adhesion is termed hard-soft electroadhesion, or EA^{[HS]}, and endures after the field is removed. Depending on the material, adhesion occurs at the anode (+), cathode (−), or both electrodes. In many cases, EA^{[HS]} can be reversed by re-applying the field with reversed polarity. Adhesion via EA^{[HS]} to AAm gels follows the electrochemical series. EA^{[HS]} arises via electrochemical reactions that generate chemical bonds between the electrode and the polymers in the gel.

### CROSS REFERENCE TO RELATED APPLICATIONS

This application claims priority under 35 U.S.C. § 119 to provisional patent application U.S. Ser. No. 63/559,821, filed Feb. 29, 2024. The provisional patent application is hereby incorporated by reference in its entirety herein, including without limitation: the specification, claims, and abstract, as well as any figures, tables, appendices, or drawings thereof.

### TECHNICAL FIELD

The subject matter of the present disclosure relates generally to adhesion between dissimilar materials and the reversibility of the adhesion. More specifically, the present disclosure is directed to systems and methods of adhesion and its reversal between hard materials, such as for example, metals and soft materials, such as for example hydrogels.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

The present disclosure concerns both soft and hard solids. Common examples of soft solids are hydrogels, which are three-dimensional networks of polymer chains swollen with water. The networks can be crosslinked by chemical (covalent) bonds or physical (non-covalent) bonds such as hydrogen-bonds. Examples of the latter include gelatin gels, which are a popular dessert (Jell-O®) in many parts of the world. Other examples of soft, aqueous materials include plant products (fruits and vegetables), aquatic animals, and the tissues in the body. Typical biological cells are soft and gel-like, with a water content around 70%. Yet, the architecture of vertebrate animals, including humans, shows the need to combine soft tissues with hard structural elements, i.e., bones, vertebrae, and the skeleton. The hard elements are needed to support the weight of the animal, provide structural integrity, and transmit force. The need to combine soft and hard elements also comes into the fore when designing soft robots or actuators. Researchers have realized that, for a robot to exert forces, soft, force-generating elements (akin to muscle) must be interfaced with stiff, load-bearing elements (akin to bone) via elements of intermediate stiffness (akin to cartilage).

Motivated by some of the points mentioned above, several researchers have attempted to adhere soft hydrogels to hard solids (e.g., metals, plastic, wood, glass). However, to achieve adhesion, typically the chemistry of either the hydrogel or the hard surface has to be modified. One common modification has been inspired by the chemistry of mussels and is via catechol groups. Mussels stick to rocky surfaces by secreting filaments rich in catechols, which form strong coordination bonds with the surfaces. Accordingly, catechols can be introduced into gel backbones to make gels adhere to hard surfaces. Other chemistries have also been exploited to induce strong gel-solid adhesion. For instance, one study functionalized acrylamide (AAm) gels with azide groups and contacted the gels with glass surfaces decorated with alkyne groups. A cycloaddition reaction between the azides and alkynes ensued, leading to strong adhesion. In most of the above cases where a gel is bonded to a hard solid, the adhesion is permanent, i.e., the two cannot be easily detached at a later time if needed. Thus, to sum up the literature, gel-solid adhesion has been achieved mostly for chemically tailored gels or surfaces, and once the gel is adhered to the solid, their adhesion is generally permanent and irreversible.

There has been interest in triggering adhesion between materials (i.e., achieving ‘adhesion on command’) via an external stimulus such as electric fields. See e.g., Borden et al., “Reversible electroadhesion of hydrogels to animal tissues for suture-less repair of cuts or tears.” Nat. Commun. 2021, 12, 4419; and Borden et al., “Universal way to ‘glue’ capsules and gels into 3D structures by electroadhesion.” ACS Appl. Mater. Interfaces 2023, 15, 17070-17077; each of which are hereby incorporated by reference in their entireties herein.

For example, a DC field can induce adhesion between a cationic and an anionic gel. When the gels are subjected to 10 V DC for ˜10 s they become stuck, and the adhesion endures after the field is removed. This gel-gel electroadhesion (EA) is believed to be due to the electrophoretic migration of polymer chains across the gel-gel interface. The applicants of the present disclosure have also shown that cationic gels can be electroadhered to animal tissues, which are known to be anionic. This gel-tissue EA again has all the above hallmarks (it is a permanent adhesion induced by 10 V DC within seconds). Interestingly, both gel-gel and gel-tissue EA can be reversed at a later point simply by placing the adhered pair under 10 V DC and reversing the polarity. Thus, EA occurs between soft, aqueous materials of opposite charge, and it is strong, durable, and reversible.

Electric fields have also been used to stick hard and soft materials, but such adhesion typically decays and disappears quickly when the field is switched off. For example, a metal can be stuck to a soft dielectric material (such as an elastomer or a non-aqueous gel) when a DC field>120 V is applied across the pair. This phenomenon is also called electroadhesion, but it is conceptually very different and has an electrostatic origin (i.e., the Johnson-Rahbek effect). When the field is turned off, the materials quickly lose this electrostatic adhesion thus, this phenomenon allows metallic grippers in a robot to pick up objects and then release them. Note that this electrostatic effect cannot lead to permanent adhesion in the absence of the field. It is believed that the only example of enduring hard-soft adhesion induced by an electric field was in the recent study of Qiu et al “Electrochemical bonding of hydrogels at rigid surfaces,” Small Methods 2022, 6, e2201132, where a hydrogel was contacted with glass as well as two iron electrodes and applied a DC field of ˜6 V for several hours. The glass (but not the electrodes) adhered to the gel, and this was attributed to the formation of iron (III) hydroxide nanoparticles at the gel-glass interface. This finding seems to be restricted to a particular choice of electrodes (and to thin gels) and so is not generalizable.

Thus, there exists a need in the art for systems and methods to electroadhere a wide variety of materials to one another without the need for complex equipment and/or methods for said electroadhesion.

### SUMMARY

Embodiments of the present disclosure are directed to systems and methods in which hard electronic conductors (e.g., metals or graphite) can be electroadhered to a range of soft aqueous materials, including hydrogels, fruit, and animal tissue. In one embodiment, two graphite slabs are placed on either side of a cylindrical hydrogel (5 cm tall) and 5 V DC is applied across the combination for ˜3 min. After this period, one of the graphite slabs is found to be strongly stuck to the hydrogel. This adhesion endures long after the field is removed (gel-graphite pairs have remained adhered for months). This phenomenon is termed herein throughout the present disclosure as “hard-soft electroadhesion” or “EA^{[HS]}” and it is conceptually different from all previous uses of the term ‘electroadhesion.’ For one or more embodiments of the present disclosure, adhesion can be achieved in just a few seconds if the gel has a high ionic conductivity. The adhesion is very strong: the strength of the adhesion is limited mostly by the strength of the gel and is shown to exceed 150 kPa.

In one or more embodiments of EA^{[HS]} described herein, numerous hard-soft material pairs are examined. On the soft side, EA^{[HS]} works with chemical gels like AAm, physical gels like gelatin and alginate, and even soft objects like fruit (bananas, apples) and animal tissue (beef, pork). Cationic, anionic, and nonionic gels can all be bonded to hard solids by this method. On the hard side, EA^{[HS]} is achieved with many metals (e.g., copper, lead, tin, nickel, iron, or zinc). Depending on the gel chemistry, adhesion occurs at the anode (+), cathode (−), both electrodes, or neither. If EA^{[HS]} is observed only to one electrode, generally it can be reversed by switching the polarity of the electrodes and re-applying the field.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to electroadhere hard materials to soft materials. The hard and soft material can be brought into contact with one another and electroadhered to one another by applying a low DC electric field (e.g., 5 V) applied for a short time (e.g., 3 min).

It is still yet a further object, feature, and/or advantage of the present disclosure to conduct electrons with the hard material. Examples of such materials comprise carbon-based materials, metals, alloys, and the like. Metals, for example, that can be adhered via EA^{[HS]} to AAm gels (all at the anode) have reduction potentials above a critical value. This correlation to the electrochemical series suggests that EA^{[HS]} is due to chemical bonds between the gel and the anode induced by electrochemical reactions.

It is still yet a further object, feature, and/or advantage of the present disclosure to conduct ions with the soft material. The soft material can comprise water and ions (salt). Examples of such materials include hydrogels as well as plant-based tissues (e.g., fruits and vegetables) and animal-based tissues (e.g., meat from cows, pigs, and chickens). Salt can be added to the soft-material to simulate the salt content in blood (e.g., around 0.9%).

It is still yet a further object, feature, and/or advantage of the present disclosure to induce adhesion at the anode (+), cathode (−), both electrodes, or neither, depending on the nature of the hard and soft materials.

It is still yet a further object, feature, and/or advantage of the present disclosure to allow adhesion to endure after the field is removed.

It is still yet a further object, feature, and/or advantage of the present disclosure to remove the adhesion by switching the polarity of the field.

It is still yet a further object, feature, and/or advantage of the present disclosure to increase adhesion strength by increasing voltage, time in the field, and/or ionic conductivity of the gel.

It is still yet a further object, feature, and/or advantage of the present disclosure to build structure(s) formed from combinations of hard materials and soft materials. The use of reversible electroadhesion EA^{[HS]} can allow some objects to be placed into physical configurations that would not otherwise be constructable without the use of welds (e.g., the Olympic rings).

EA^{[HS]} can be used in a wide variety of applications. Overall, this phenomenon is remarkable in its simplicity and wide applicability, and thus the discovery of its existence and applications of same is quite significant. For example, EA^{[HS]} can create new hybrid materials, thus enabling applications in robotics, energy storage, and biomedical implants. Interestingly, EA^{[HS]} can even be achieved underwater, where typical adhesives cannot be used.

Methods can be practiced which facilitate use, manufacture, assembly, maintenance, and repair of EA^{[HS]} which accomplish some or all of the previously stated objectives.

The use of EA^{[HS]} can be incorporated into systems or structures which accomplish some or all of the previously stated objectives.

According to some aspects of the present disclosure, a method of hard-soft electroadhesion (EA^{[HS]}), comprises conducting electrons with a hard material; conducting ions with a soft material; and applying an electric field so as to adhere the hard material and the soft material to one another. The adhesion of the soft material and hard material endures after the electric field is removed.

According to some additional aspects of the present disclosure, the method further comprises adding a salt to the soft material.

According to some additional aspects of the present disclosure, the method further comprises unsticking the hard material from the soft material by reversing the electric field.

According to some additional aspects of the present disclosure, a strength of the electric field is preferably no less than one volt (1 V) and no more than twenty volts (20 V), more preferably between three volts (3 V) and ten volts (10 V), and most preferably being about five volts (5 V).

According to some additional aspects of the present disclosure, the electric field is preferably applied for a duration of time of no less than thirty seconds (30 sec) and no more than fifteen minutes (15 min), more preferably between one minute (1 min) and five minutes (5 min), and most preferably about three minutes (3 min).

According to some additional aspects of the present disclosure, the hard material is selected from the group consisting of: a carbon-based mineral, a metal, and an alloy. The hard material can comprises titanium, stainless steel, or nitinol, which can be good materials for implants within a body, such as where the soft material is animal tissue. The carbon-based mineral could comprise graphite.

According to some additional aspects of the present disclosure, the soft material is a hydrogel that comprises water. Optionally, the soft material can comprise a reduction potential greater than 0. Optionally, the soft material can comprise a cylindrically shaped form.

According to some additional aspects of the present disclosure, the soft material comprises plant material.

According to some additional aspects of the present disclosure, the method further comprises correlating pull off strength to one or more of a duration in which the electric field is applied, a strength of the electric field, or a salt concentration in the soft material.

According to some additional aspects of the present disclosure, the method further comprises selecting to induce adhesion at an anode or a cathode based on a type of the soft material.

According to some additional aspects of the present disclosure, the soft material is capable of adhering to both an anode and a cathode.

According to some other aspects of the present disclosure, a reversible system comprises a hard material capable of conducting electrons; a soft material capable of conducting ions; and a battery that can apply an electric field such that the hard material and the soft material adhere to one another.

According to some additional aspects of the present disclosure, the reversible system is located underwater.

According to some additional aspects of the present disclosure, the reversible system comprises an armor, such as to help protect animals without hard skeletons, or an implant for a living organism, such as for humans that are at risk of injuries or further injuries.

According to some additional aspects of the present disclosure, the reversible system comprises a structure formed from at two hard materials adhered to one or more soft materials.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1B shows reversible electroadhesion of graphite to an acrylamide (AAm) hydrogel. Photos and schematics are shown for each case. First, FIG. 1A shows graphite slabs placed on either end of the AAm gel cylinder (dyed yellow) and 5 V DC is applied for 3 min (A1). The graphite anode (+) becomes strongly adhered to the gel (A2), allowing the pair to be lifted up in mid air. FIG. 1B shows the graphite slabs are again contacted with the gel, and the polarity is reversed, i.e., the adhered slab is now the cathode (−). Upon applying 5 V DC for 3 min, the adhered slab is detached while the bottom one (new anode) is now adhered to the gel. Scale bars are 1 cm.

FIGS. 2A-2F show factors that affect the adhesion strength achieved by EA^{[HS]} between graphite and AAm gels. The pull-off adhesion strength is shown in each graph. Mean values are plotted and the error bars represent standard deviations from n≥3 measurements. FIG. 2A shows results from varying the voltage across the gel. FIG. 2B shows results from varying the time over which the voltage is applied. FIG. 2C shows results from varying the concentration of salt (NaCl) in the gel. FIG. 2D shows results from varying the concentration of monomer (AAm) used to make the gel. The filled symbols correspond to 30 s of applying the voltage and the open symbols to 3 min. FIG. 2E shows the EA^{[HS]} values from FIG. 2D for 20 and 50% AAm are compared with the values for contact adhesion. EA^{[HS]} is much stronger. To illustrate the strength of EA^{[HS]}, FIG. 2F shows that a graphite-gel pair (gel is 20% AAm) can support an additional weight of 100 g.

FIGS. 3A-3C show pull-off testing and failure modes. FIG. 3A shows a schematic of the pull-off testing setup. Tests are conducted using a rheometer in a parallel-plate geometry. A given gel and graphite are adhered by EA^{[HS]}. The gel is then glued onto the top plate and the graphite is glued onto the bottom plate. The top plate is then pulled upwards while the stress is monitored. The stress at the point of failure is the adhesion strength induced by EA^{[HS]}. FIG. 3B shows when the adhesion strength is low (below ˜20 kPa), the failure mode is adhesive, i.e., the gel neatly detaches as a whole from the graphite. This is shown by the photo and the schematic. FIG. 3C shows when the adhesion strength is high (above ˜30 kPa), the failure mode is cohesive, i.e., the gel breaks in the middle. This is shown by the photo as well as the schematic. Cohesive failure indicates that the adhesion is stronger than the strength of the gel.

FIGS. 4A-4B show rheological properties of acrylamide (AAm) gels studied with regard to their adhesion to graphite via EA^{[HS]} in FIG. 2D. FIG. 4A shows data from dynamic rheology for the elastic modulus G′ and the viscous modulus G″ as functions of the angular frequency ω. All samples show the rheology characteristic of a gel, with G′ being independent of ω. Each gel can thus be characterized by its value of G′, which is the gel modulus. FIG. 4B plots the gel modulus vs. AAm concentration, showing a power-law relationship. At low AAm (10%), the gel is soft (modulus ˜7 kPa), whereas at high AAm (50%), the gel is very stiff (modulus ˜150 kPa).

FIG. 5 shows adhesion results at the anode for various hard materials to AAm gels by EA^{[HS]}. The results are shown in an electrochemical series with the standard reduction potential E° for each material. Photos are shown for each case. Strips of each material are placed on either end of a cylindrical AAm gel and 5 V DC is applied for up to 15 min. Adhesion occurs only to the anode (+). Materials that adhere are all on the right side of the series, i.e., their E°>−0.2 V, indicating that they are relatively inert. In all these cases, the material-gel pair can be lifted up in the air. Materials that do not adhere have more negative E°, indicating that they are more reactive (easily oxidized). Scale bars are 1 cm.

FIGS. 6A-6C show adhesion results for graphite to various gels by EA^{[HS]}. The results are shown through photos. Gels are imbued with dyes and are thus color-coded as follows: nonionic gels in yellow, anionic gels in blue and cationic gels in pink. Strips of graphite are placed on either end of a cylindrical gel and 5-10 V DC is applied for 15 min. FIG. 6A shows gels that adhere only to the anode (+). FIG. 6B shows gels that adhere only to the cathode (−). FIG. 6C shows gelatin is the only gel that adheres to both electrodes. Scale bars are 1 cm.

FIGS. 7A-7C show adhesion results for graphite to various plant and animal tissues by EA^{[HS]}. The results are shown through photos. Strips of graphite are placed on either end of a given soft material and 5 V DC is applied for 15 min. FIG. 7A shows tissues that adhere only to the anode (+). FIG. 7B shows tissues that adhere only to the cathode (−). FIG. 7C shows tissues that adhere to both electrodes.

FIGS. 8A-8C show use of EA^{[HS]} to adhere hard and soft materials in various configurations. FIG. 8A shows a thin AAm gel is used as an adhesive between two Cu sheets, labeled Cu1 and Cu2. AAm is first stuck to Cu1 using graphite as a counter electrode and then the AAm is stuck to Cu2. FIG. 8B shows a ring of alternating gelatin gels and graphite strips is bonded together in a single step. This is possible because gelatin adheres to graphite at both electrodes. FIG. 8C shows a robust chain of eight different hard materials connected by AAm and QDM gels.

FIGS. 9A-9B show probing the mechanism for EA^{[HS]} using FTIR. Spectra are shown for the cases of graphite-AAm gel (FIG. 9A) and graphite-agarose gel (FIG. 9B). AAm adheres to graphite at the anode by EA^{[HS]}, whereas agarose does not adhere to either electrode. Gel slices next to the electrodes or in the bulk are analyzed; see FIGS. 10A-10B, infra, for details. No chemical changes are detected in FIG. 9B from the IR spectra. In FIG. 9A, only the gel slice near the anode (denoted as G/+, purple curve) shows evidence of new bonds. A close-up of the key peaks is provided in FIGS. 11A-11B, infra.

FIGS. 10A-10B show gel samples prepared for analysis by FTIR. A tall cuboidal gel is contacted with graphite slabs on either end and 5 V DC is applied for 5 min. Next, thin slices of the gel are made at different locations. The slice in the middle is denoted as G^{bulk}. The slice next to the anode is G/+ and that next to the cathode is G/−. These gel slices are then analyzed by FTIR-ATR. Two gels are considered. FIG. 10A shows a gel of acrylamide (AAm), which adheres by EA^{[HS]} to the anode. In this case, the anode slice (G/+) has some graphite clinging to it (Photo A1) and this graphite cannot be removed by washing with water (Photo A2). The cathode slice (G/−) also initially appears to have some graphite stuck to it (Photo A1), but this graphite is easily washed away with water. The G^{bulk} slice is clear. In FTIR (see FIG. 9A), only G/+ shows evidence of new bonds. FIG. 10B shows a gel of agarose, which does not adhere to either electrode. In this case, all the gel slices are clear, and no new bonds are detected in any of them by FTIR (see FIG. 9B).

FIGS. 11A-11B show additional FTIR data and analysis for the graphite-AAm gel pair. FIG. 11A shows a close-up of data shown in FIG. 9A, highlighting the peaks in the vicinity of 1600 cm⁻¹. For the AAm gel slice near the anode (G/+), the new peak at 1582 cm⁻¹ indicates new bonds induced by EA^{[HS]} between graphite and AAm. FIG. 11B shows data for the G/+ gel slice is compared to two other cases. First, the gel slice near the cathode (G/−) has a spectrum that is closer to the AAm in the bulk. Next, an AAm gel was adhered at the anode, then the polarity was reversed to detach the gel. The slice at this electrode interface is the top curve and it shows a more complex spectrum. Importantly, the peak at 1582 cm⁻¹ is no longer present.

FIG. 12 shows hypothesized bonds induced between different gels and graphite by EA^{[HS]} and an accompanying schematic for the adhesion of an AAm gel to graphite via the pertinent bonds.

FIGS. 13A-13E show an electrogripper based on EA^{[HS]}. FIG. 13A shows a graphite slab is stuck to a glass rod and connected to DC power. Two pieces of Al foil serve as the counter electrodes. An AAm gel to be picked up is placed on one foil. FIG. 13B shows the graphite slab is contacted with the gel and serves as the anode (+). 5 V is applied for 5 s. FIG. 13C shows the gel is stuck to the graphite slab and is picked up. FIG. 13D shows the gripper moves the gel to the other foil. The graphite is now made the cathode (−) and 5 V is applied for 15 s. FIG. 13E shows the gel detaches from the graphite and is dropped off.

FIGS. 14A-14B show a primary battery created by EA^{[HS]}. FIG. 14A shows the battery has Cu and Zn strips as electrodes flanking a hybrid AAm/QDM gel (with 1% NaCl in it) as the electrolyte. The metal strips are adhered to the gel using EA^{[HS]}. FIG. 14B shows that under open-circuit conditions, with Cu as the cathode (+) and Zn as the anode (−), the battery delivers a potential of ˜0.9 V (B1) and this remains stable after 6 h (B2).

FIGS. 15A-15E show load-bearing assembly fabricated by EA^{[HS]}. FIG. 15A shows four flexible gel pillars are electroadhered between two graphite slabs. Different weights are loaded on the top. FIG. 15B shows 20 g causes minimal compression. FIG. 15C shows 50 g causes the pillars to buckle and bend. FIG. 15D shows 100 g makes the pillars buckle until the top slab is compressed down to the bottom one. FIG. 15E shows when the 100 g load is removed, the assembly retracts to its original state.

FIGS. 16A-16C shows underwater adhesion of metal and gel by EA^{[HS]}. FIG. 16A shows a Cu sheet and an AAm gel are contacted underwater. With the Cu as anode and graphite as the cathode (not shown), EA^{[HS]} is induced between the metal and the gel. FIG. 16B shows after the field is switched off, the pair remain adhered. FIG. 16C shows a second Cu sheet is adhered on the opposite side of the gel by EA^{[HS]}. The gel thus serves as an underwater adhesive between the two metal sheets.

FIG. 17 shows that in some cases, alternating current (AC) enables adhesion (EA^{[HS]}) of a metal to a gel while direct current (DC) does not.

FIGS. 18A-18B show that in some cases, if adhesion by DC of a metal to a gel or soft material is not observed, the reason could be a low current. If so, increasing the voltage, and thereby the current, can enable EA^{[HS]}.

FIGS. 19A-19B show that EA^{[HS]} can be used to adhere graphite and metals to soft materials that are dry provided the soft material is ionically conductive. Likewise, EA^{[HS]} can be used to adhere to soft gels that contain no water, but a non-aqueous polar liquid, provided the gels are ionically conductive.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

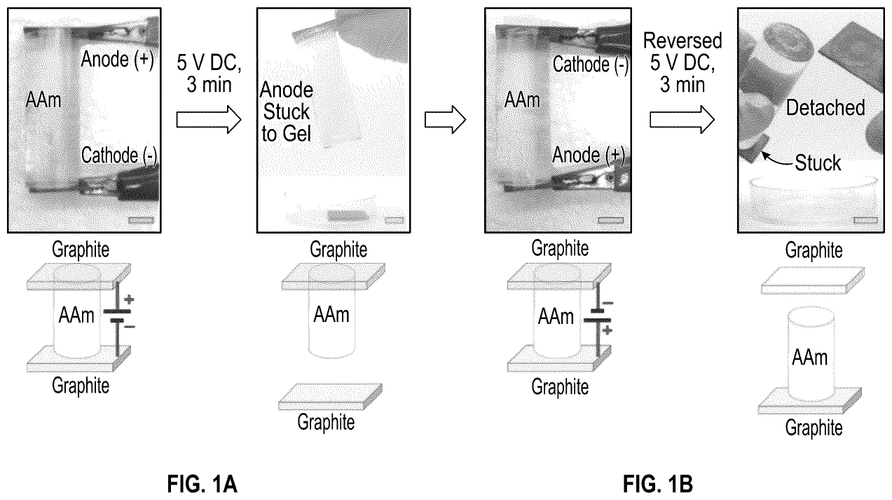

The present disclosure evidences that hard electronic conductors (e.g., metals or graphite) can be electroadhered to a range of soft aqueous materials, including hydrogels, fruit, and animal tissue. These hard-soft combinations improve on earlier works to the present inventors that involve gels and tissues. Beneficially, the experiments and tests discussed herein do not require complex setups to conduct, and therefore can be more easily replicated and verified by others. For example, two graphite slabs can be placed on either side of a cylindrical hydrogel (5 cm tall) and 5 V DC is applied across the combination for ˜3 min. After this period, one of the graphite slabs is found to be strongly stuck to the hydrogel, as shown in FIGS. 1A-1B. This adhesion endures long after the field is removed (gel-graphite pairs have remained adhered for months). As used herein, this phenomenon is termed “hard-soft electroadhesion,” or EA^{[HS]}. EA^{[HS]} is conceptually different from all previous uses of the term ‘electroadhesion’. Adhesion can be achieved in just a few seconds if the gel has a high ionic conductivity. The adhesion is very strong: the strength of the adhesion is limited mostly by the strength of the gel and is shown to exceed 150 kPa.

Over the course of the present disclosure which regards EA^{[HS]}, numerous hard-soft material pairs are examined. On the soft side, EA^{[HS]} works with chemical gels like Aam; physical gels like gelatin and alginate; and even soft objects like fruit (e.g., bananas, apples) and animal tissue (e.g., beef, pork). Cationic, anionic, and nonionic gels can all be bonded to hard solids by this method. On the hard side, EA^{[HS]} is achieved with many metals (e.g., copper, lead, tin, nickel, iron, or zinc). Depending on the gel chemistry, adhesion occurs at the anode (+), cathode (−), both electrodes, or neither. If EA^{[HS]} is observed only to one electrode, generally it can be reversed by switching the polarity of the electrodes and re-applying the field. With regard to the mechanism behind EA^{[HS]}, the present disclosure shows that it arises due to electrochemical reactions that generate bonds between the hard electrode and the polymers in the gel network. Hybrid materials created by EA^{[HS]} highlight its utility in robotics, energy storage, biomedical implants, and surgery.

### Reversible Adhesion of Graphite to AAm Gels

The adhesion between graphite and acrylamide (AAm) gels are shown in FIGS. 1A-1B. The AAm gel was made by free-radical polymerization using 20% AAm, with N,N′-methylenebis (acrylamide) (BIS) (1.5% of the AAm) as the crosslinker. Salt (1% NaCl) was added to the gel for ionic conductivity. Gels were typically prepared in the shape of a cylinder (2 cm diameter, 5 cm tall, total weight ˜30 g). This geometry allows one to easily check if a hard solid was strongly adhered to the gel. As shown in FIG. 1A, when adhesion occurs, the solid is able to hold the gel in mid-air. The graphite slabs were cut from a larger piece to a size of 3×2×0.2 cm. As a control, when a graphite slab and the AAm gel were pressed into contact, there was no adhesion and the two could be separated right away. Next, graphite slabs are placed on the top and bottom of the gel (FIG. 1A, left-side panel) and these are connected to a DC power supply. The graphite slabs thus serve as electrodes, i.e., one is the anode, connected to the positive terminal of the power supply, and the other is the cathode, connected to the negative terminal. With this setup, 5 V DC is applied across the gel for three minutes (3 min). After the DC field is stopped, the graphite anode is found to be strongly adhered to the AAm gel. The right-side panel of FIG. 1A shows the anode lifting up the gel in mid-air.

The above result was surprising and unexpected. Qualitatively, a strong adhesion had been induced between the gel and the slab. If it was tried to wrench apart the gel and the slab, typically the gel would break and pieces of the gel would be left behind on the graphite surface. The adhesion persisted indefinitely so long as the gel did not lose water (e.g., if the graphite-gel pair was stored in a closed container). Such adhered pairs were preserved in the lab for months and they still remain adhered. If the gel is left to dry in air, it shrinks considerably and then the adhesion to the slab weakens gradually due to a size mismatch.

A continuation of the experiment between graphite and AAm gel is shown in FIG. 1B. The adhered graphite-gel pair is used and placed back with the unadhered graphite slab on the other side. Then, the DC field in the reverse direction, i.e., the polarity is switched, as shown in the left-side panel of FIG. 1B. The previously adhered graphite anode is now the cathode (−) while the other graphite slab serves as the new anode (+). With this configuration, a five volt direct current (5 V DC) is applied for three minutes (3 min). After the DC field is stopped, the previously adhered graphite is found to have detached, as shown in the right-side panel of FIG. 1B. Conversely, the previously unadhered slab is now stuck to the gel. These results imply that graphite adheres to AAm gels if it is the anode in a DC circuit, but not if it is the cathode. Moreover, this implies that the adhesion can be reversed as and when desired by applying the DC field with reversed polarity.

### Factors That Affect the Adhesion Strength

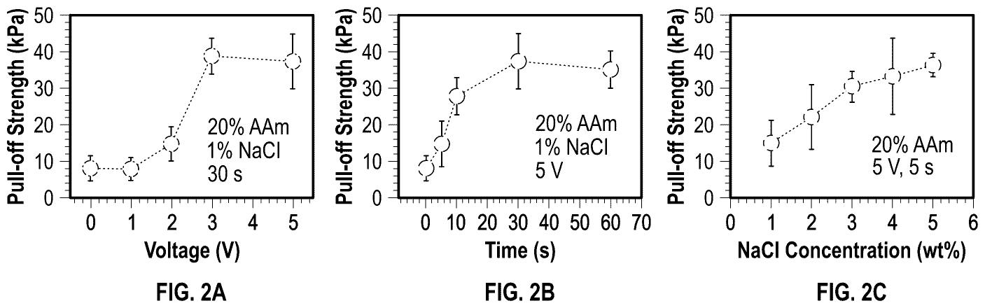

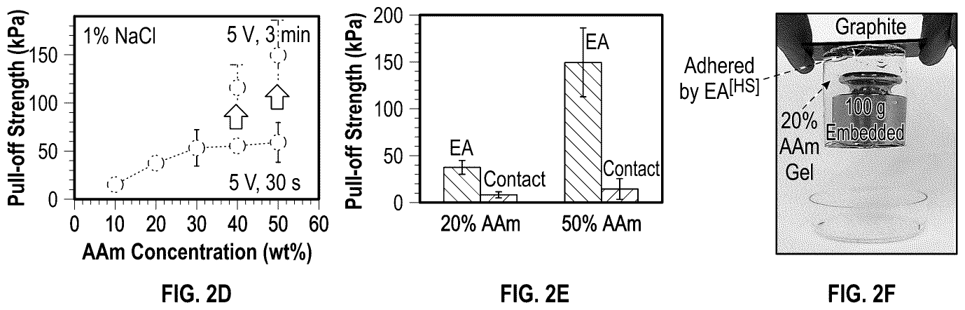

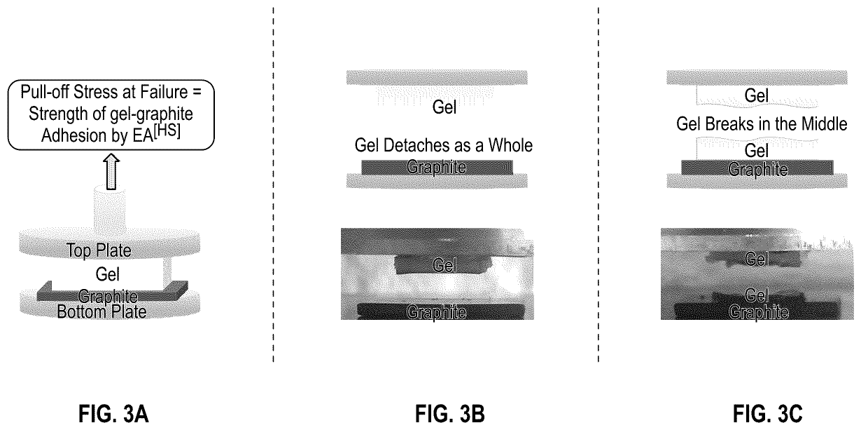

The phenomenon shown by FIGS. 1A-1B is termed as “hard-soft electroadhesion” or EA^{[HS]}. To determine main factors that affect EA^{[HS]}, pull-off testing was conducted on adhered graphite-AAm pairs, and the results are presented in FIGS. 2A-2F. The gel pieces for these tests were made as cuboids with a base of 1×1 cm and a height of 1.6 cm. The test setup is shown in FIG. 3A. From the experiments, the pull-off adhesion strength was obtained, which is the tensile stress required to separate the gel from the graphite slab (i.e., the stress at break).

The effect of varying the voltage on graphite-AAm EA^{[HS]} is presented in FIG. 2A. The DC voltage was varied from zero to five volts (0 to 5 V) across the AAm gels. The gel composition was fixed at the one used in FIGS. 1A-1B and in each case, the voltage was applied for thirty seconds (30 s). Below 1 V, the adhesion strength is negligible (i.e., it is comparable to contact adhesion). At two volts (2 V), EA^{[HS]} is noticeably stronger than contact adhesion. In this case, when the gel is pulled off from the graphite, an adhesive failure occurs, i.e., the gel and graphite separate at their interface (FIG. 3B). This mode of failure was consistently observed when the adhesion strength was low (<20 kPa). When the voltage is increased to three volts (3 V), the adhesion strength increases to 40 kPa. In this case, a cohesive failure occurs, i.e., the failure occurs in the middle of the gel (FIG. 3C). Such failure was always observed when the adhesion strength was high (>30 kPa). It indicates that the adhesion is so strong that it exceeds the gel strength. Consequently, the measured adhesion strength due to EA^{[HS]} levels out as the voltage is increased above three volts (3 V).

Next, the time over which the electric field was applied was varied, as shown in FIG. 2B. The AAm gel was the same as above and the voltage was fixed at five volts (5 V). Over the first thirty seconds (30 s), the adhesion of the gel to graphite strengthens with increasing time, indicating that EA^{[HS]} accumulates as the voltage is applied. Thereafter, the adhesion plateaus. An adhesive failure for the initial points (<20 kPa in adhesion strength) is observed and a cohesive failure for the subsequent ones. The results indicate that for a 1.6 cm-tall gel sample, strong adhesion can be achieved within a minute of applying the field. For the taller gels studied in FIGS. 1A-1B (five centimetres (5 cm) height), a longer time (˜3 min) was needed to achieve strong EA^{[HS]}. This is why the field was applied for a time of three minutes (3 min) in FIGS. 1A-1B.

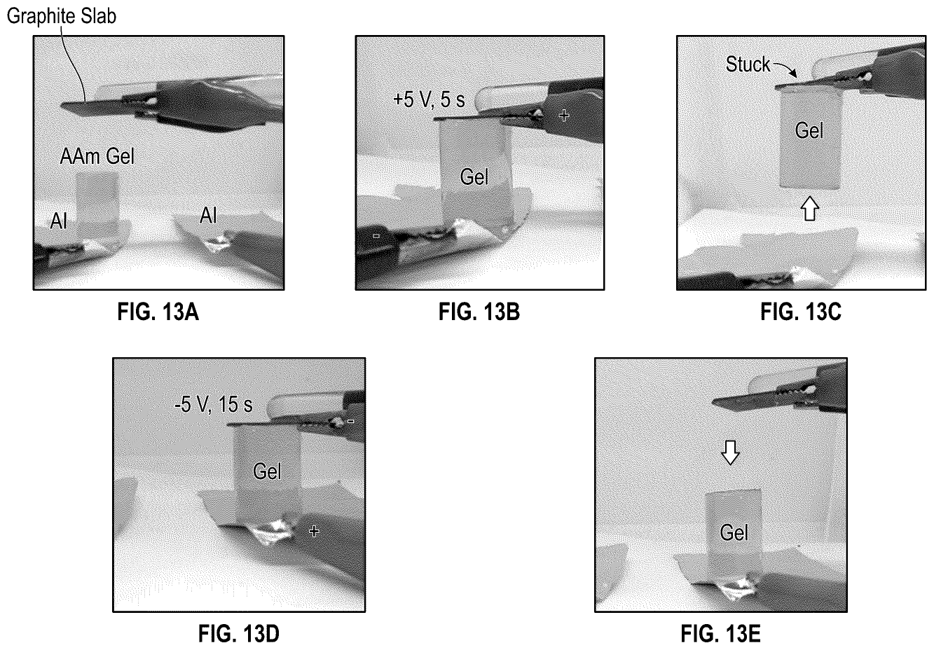

The salt (electrolyte) concentration in the gel also plays an important role in EA^{[HS]}, as shown in FIG. 2C. For a gel that is nonionic like AAm, in the absence of salt, the ionic conductivity is very low. It is only with the addition of salt that the gel becomes conductive. A conductive gel is needed to complete the DC circuit. As the salt in the gel increases, the current through the circuit increases. This has an effect on the adhesion strength, as shown in FIG. 2C. For these experiments, the same 20% AAm gel was used and applied a voltage of five volts (5 V) for five seconds (5 s). NaCl was used as the salt and its concentration in the gel was varied. The results show that the adhesion strength of the gel to graphite increases monotonically with increasing NaCl. A key corollary of this result is that if the gel has high salt, it can be stuck by EA^{[HS]} to hard solids in a very short time. For example, a tall (5 cm) AAm gel with 15% NaCl can be adhered strongly to graphite in only five seconds (5 s). This result is discussed in greater detail with respect to FIGS. 13A-13E, infra.

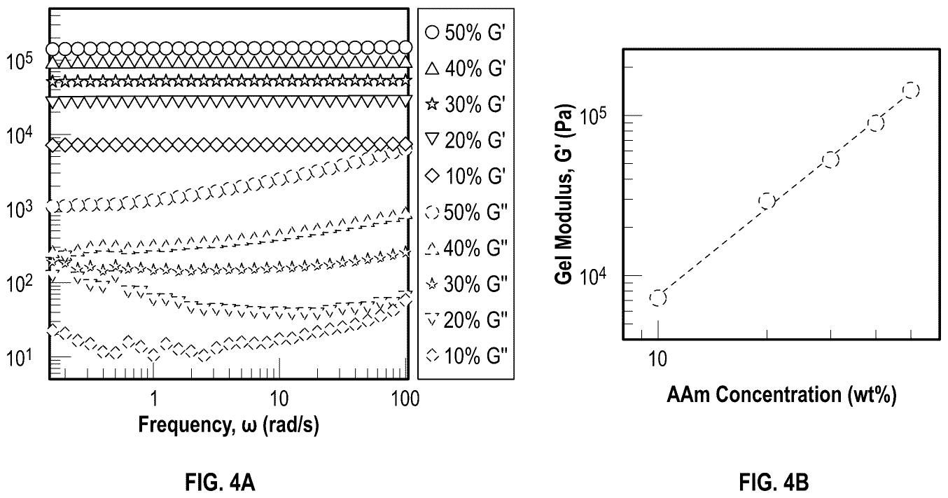

The effect of gel properties on EA^{[HS]} was also studied. A first key variable is the concentration of polymer chains, which can be altered via the AAm monomer content used during synthesis. The gels so far all had 20% AAm with the crosslinker BIS at 1.5% of the AAm. The same BIS: AAm ratio was kept and varied the AAm from 10 to 50%. As the AAm increased, the gels transformed from soft to stiff, as shown in the rheological data of FIGS. 4A-4B. Each gel was loaded with 1% NaCl and their adhesion to graphite induced by five volts direct current (5 V DC) for thirty seconds (30 s). FIG. 2D shows that the adhesion strength increases with polymer concentration. The values for the 10%, 20%, and 30% AAm gels correspond to the maximum value of adhesion strength at five volts (5 V). That is, the time of thirty seconds (30 s) was sufficient for these gels to reach a plateau in adhesion strength vs. time, as found in FIG. 2B. For the 40 and 50% AAm gels, increasing the time in the field beyond thirty seconds (30 s) to three minutes (3 min) increased the adhesion strength (open symbols in FIG. 2D). For the 50% AAm gel, the bar graph in FIG. 2E contrasts the EA^{[HS]} adhesion strength, which is ˜150 kPa, with the value for contact adhesion, which is ˜15 kPa. The comparison implies that EA^{[HS]} can be ten times (10x) the strength of contact adhesion if the gel is strong. FIG. 2F shows a one hundred gram (100 g) weight embedded in a 20% AAm gel and then stuck to graphite by EA^{[HS]}. The pair can be held up vertically, which vividly shows the high strength of EA^{[HS]}.

### Adhesion to Various Hard Materials

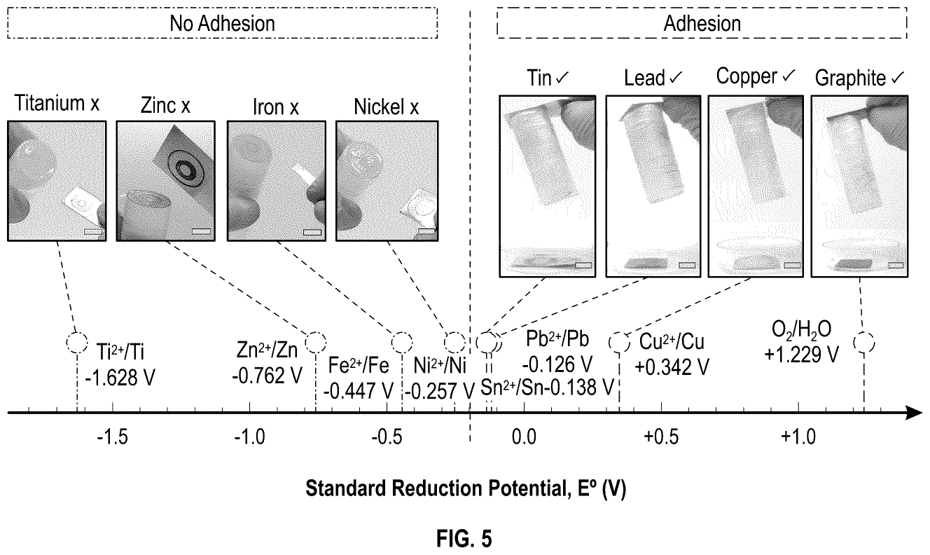

Apart from graphite, other hard materials can undergo EA^{[HS]}. Tests were performed with AAm gels and different metals (FIG. 5). The gels are similar to those in FIGS. 1A-1B. 20% AAm with 1% NaCl and in the form of a 5-cm-tall cylinder. Each metal is in the form of rectangular strips (˜3×1 cm) with 0.2 to 0.8 mm thickness. The metal strips are placed on either side of a gel cylinder and five volts direct current (5 V DC) is applied for up to fifteen minutes (15 min). All metals showed negligible contact adhesion with the gel, i.e., there was no adhesion in the absence of the field. Upon applying the field, EA^{[HS]} is induced with several metals on the anode (+) side, but there is no adhesion to the cathode (−) side. When adhesion occurs, the metal-gel pair can be lifted up in the air, and this adhesion persists afterward.

The results of anodic adhesion to AAm are shown in FIG. 5. Graphite, copper (Cu), lead (Pb) and tin (Sn) all adhere to AAm gels, while nickel (Ni), iron (Fe), zinc (Zn) and titanium (Ti) do not. All the above were arranged in an electrochemical series, whereupon a pattern emerges. Metals that do not adhere to AAm have negative standard reduction potentials E°, indicating that they are more reactive, i.e., they easily lose electrons and thereby get oxidized. Conversely, materials that do adhere to AAm are relatively inert. These have positive (or not so negative) E°, with a cut-off value for adhesion being around −0.2 V. The correlation between adhesion and the electrochemical series suggests that EA^{[HS]} arises due to electrochemical reactions at the interface. At the anode, where the half-reaction is oxidative, the DC field causes the gel to react electrochemically with the inert metal (instead of electrolyzing the metal into cations). The result from FIG. 5 is for AAm gels only. Some metals on the left of FIG. 5 like Zn and Fe do undergo EA^{[HS]} to other gels, as will be discussed below.

### Adhesion to Various Hydrogels

Next, other hydrogels can be stuck by EA^{[HS]} to hard solids. For example, graphite was tested along with gels of different chemistry. Some were chemical gels made by free-radical polymerization (similar to AAm but with different monomers)—the crosslinks in these gels are covalent bonds. Others were physical gels, e.g., gels of polysaccharides or proteins where the crosslinks are physical, non-covalent bonds (e.g., ionic or hydrogen-bonds). The geometry was the same as in FIGS. 1A-1B: each gel in the form of a 5-cm-tall cylinder, while graphite was in the form of thin slabs. Five to ten volts direct current (5-10 V DC) was applied for up to fifteen minutes (15 min) and adhesion was assessed visually as shown in FIGS. 1A-1B and FIG. 5.

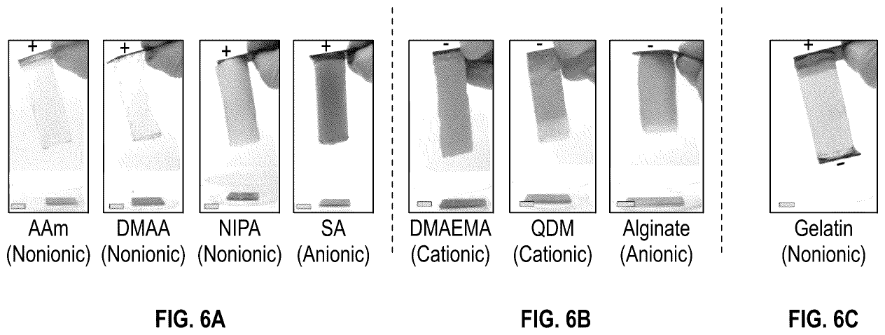

The results for graphite-gel adhesion are presented in FIGS. 6A-6C, where EA^{[HS]} can be seen in many, but not all, cases. The results are quite complex. The gels were color-coded based on their ionic nature (nonionic, anionic, cationic) using traces of water-soluble dyes. The same results, along with those from FIG. 5, are also shown in tabular form in Table 1A. Tables 1A-1C show results from EA^{[HS]} studies with various combinations of hard and soft materials. The results shown include those shown in FIG. 5 and FIGS. 6A-6C.

TABLE 1A
Adhesion of AAm gels to various metals.

| Hydrogel | Electrode | Anodic (+) | Reversal of | Cathodic (−) | Reversal of | |
|---|---|---|---|---|---|---|
| material | material | adhesion | adhesion | adhesion | adhesion | |
| AAm | Graphite | ◯ | ◯ | X | — | Adhesion |
| | (C) | | | | | to inert |
| AAm | Copper | ◯ | X | X | — | metals at |
| | (Cu) | | | | | the anode |
| AAm | Lead (Pb) | ◯ | X | X | — | |
| AAm | Tin (Sn) | ◯ | ◯ | X | — | |
| AAm | Nickel | X | — | X | — | No |
| | (Ni) | | | | | adhesion |
| AAm | Iron (Fe) | X | — | X | — | to reactive |
| AAm | Zinc (Zn) | X | — | X | — | metals |
| AAm | Titanium | X | — | X | — | |
| | (Ti) | | | | | |

TABLE 1B
Adhesion of graphite to various gels.

| Hydrogel | Electrode | Anodic (+) | Reversal of | Cathodic (−) | Reversal of | |
|---|---|---|---|---|---|---|
| material | material | adhesion | adhesion | adhesion | adhesion | |
| AAm | Graphite | ◯ | ◯ | X | — | Adhesion |
| (nonionic) | (C) | | | | | only to |
| DMAA | Graphite | ◯ | ◯ | X | — | anode, |
| (nonionic) | (C) | | | | | reversible |
| NIPA | Graphite | ◯ | ◯ | X | — | |
| (nonionic) | (C) | | | | | |
| SA | Graphite | ◯ | ◯ | X | — | |
| (anionic) | (C) | | | | | |
| DMAEMA | Graphite | X | — | ◯ | ◯ | No |
| (cationic) | (C) | | | | | adhesion |
| QDM | Graphite | X | — | ◯ | ◯ | to reactive |
| (cationic) | (C) | | | | | metals |
| Alginate | Graphite | X | — | ◯ | ◯ | |
| (anionic) | (C) | | | | | |
| Gelatin | Graphite | ◯ | X | ◯ | X | Adhesion |
| (nonionic) | (C) | | | | | to both |
| HEMA | Graphite | X | — | X | — | No |
| (nonionic) | (C) | | | | | adhesion |
| Agarose | Graphite | X | — | X | — | |
| (nonionic) | (C) | | | | | |
| PVA | Graphite | X | — | X | — | |
| (nonionic) | (C) | | | | | |

TABLE 1C
Adhesion of QDM gels to various metals.

| Hydrogel | Electrode | Anodic (+) | Reversal of | Cathodic (−) | Reversal of | |
|---|---|---|---|---|---|---|
| material | material | adhesion | adhesion | adhesion | adhesion | |
| QDM | Graphite | X | — | ◯ | ◯ | All adhere |
| | (C) | | | | | to cathode |
| QDM | Copper | X | — | ◯ | X | only |
| | (Cu) | | | | | |
| QDM | Lead (Pb) | X | — | ◯ | X | |
| QDM | Tin (Sn) | X | — | ◯ | X | |
| QDM | Nickel | X | — | ◯ | X | |
| | (Ni) | | | | | |
| QDM | Zinc (Zn) | X | — | ◯ | X | |
| QDM | Iron (Fe) | X | — | ◯ | X | |

First, the gels in FIG. 6A all adhere to graphite only at the anode (+). These include AAm and other chemical gels made from the acrylic acid derivatives N,N-dimethyl-acrylamide (DMAA), N-isopropylacrylamide (NIPA), and sodium acrylate (SA). AAm, DMAA, and NIPA gels are nonionic, whereas SA is anionic.

Next, the gels in FIG. 6B all adhere to graphite only at the cathode (−). These include two cationic gels made by free-radical polymerization of the monomers [(2-methacryloyloxy) ethyl] tri-methylammonium chloride (QDM) and 2-(dimethylamino) ethyl methacrylate (DMAEMA). Cathodic adhesion also occurs with gels of the anionic polysaccharide alginate, which is made by crosslinking sodium alginate with divalent calcium (Ca²⁺) cations. Thus, both cationic and anionic gels adhere to cathodes. In the case of QDM gels, in addition to graphite, several metals (Cu, Pb, Sn, Ni, Fe and Zn) all adhered at the cathode (Table 1C). Note that this includes metals with both positive and negative reduction potentials.

Next comes the curious case of gelatin, as shown in FIG. 6C. Gelatin is a denatured form of the protein collagen and forms thermoreversible gels in water driven by hydrogen-bonding of the protein chains into triple helices at crosslinking points. Gelatin undergoes EA^{[HS]} to graphite at both the cathode and the anode. Gelatin is the only gel in the present disclosure that shows this behavior. Because gelatin adheres to both electrodes, this adhesion cannot be reversed by re-applying the field with reversed polarity.

The last category of gels is those that do not stick to graphite at either the anode or cathode. Gels in this category include the nonionic chemical gel made from 2-hydroxyethyl methacrylate (HEMA) and two other nonionic physical gels: those of the synthetic polymer poly (vinyl alcohol) (PVA) and the polysaccharide agarose. The fact that some gels do not adhere to hard materials is an important point to note. This means that EA^{[HS]} is not due to a simple, universal reaction between water and any solid surface. EA^{[HS]} depends on the chemistry of both the gel and the hard material.

### Adhesion to Animal and Plant Tissues

Apart from hydrogels, there are other soft materials that can be adhered to hard ones by EA^{[HS]}. This point was explored with a variety of animal and plant-based soft materials, especially those that are available as edible foods. Adhesion was attempted to graphite slabs using five volts direct current (5 V DC) for up to fifteen minutes (15 min). In the cases of fruit or vegetables, the sample was cut open and the graphite was contacted with the fleshy interior. The outer skins of many fruits are hydrophobic and may have negligible ionic conductivity.

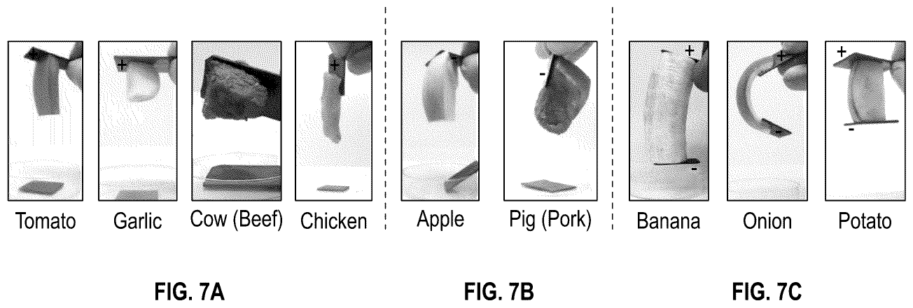

The results on EA^{[HS]} with biological tissues are interesting, but again rather complex, as shown in FIGS. 7A-7C. Some tissues adhere to graphite only at the anode (+), as shown in FIG. 7A, and these include vegetables (tomato, garlic) as well as tissues from animals: cow muscle (beef shank) and chicken muscle (segment from the thigh). Some others adhere to graphite only at the cathode (−), as shown in FIG. 7B, and these include fruit (apple) and pig muscle (pork shoulder). It is generally recognized that animal cells and tissues have an anionic character, but as can be seen here, the animal tissues tested herein show wide differences in EA^{[HS]}. Three of the plant materials were tested (banana, onion, and potato) and adhered to both electrodes, similar to gelatin gels, as shown in FIG. 7C. Lastly, there were several plant-based materials that did not adhere to either electrode, including grape, blueberry, raspberry, cucumber, orange and pear.

The results in FIGS. 5A-5C, FIGS. 6A-6C, and FIGS. 7A-7C indicate that EA^{[HS]} works with a range of both hard and soft materials. Generally, the hard material has to be an electronic conductor, which includes graphite and metals. Generally, the soft material has to be an ionic conductor, which means it has to contain water and salt.

### Adhesion in Various Configurations

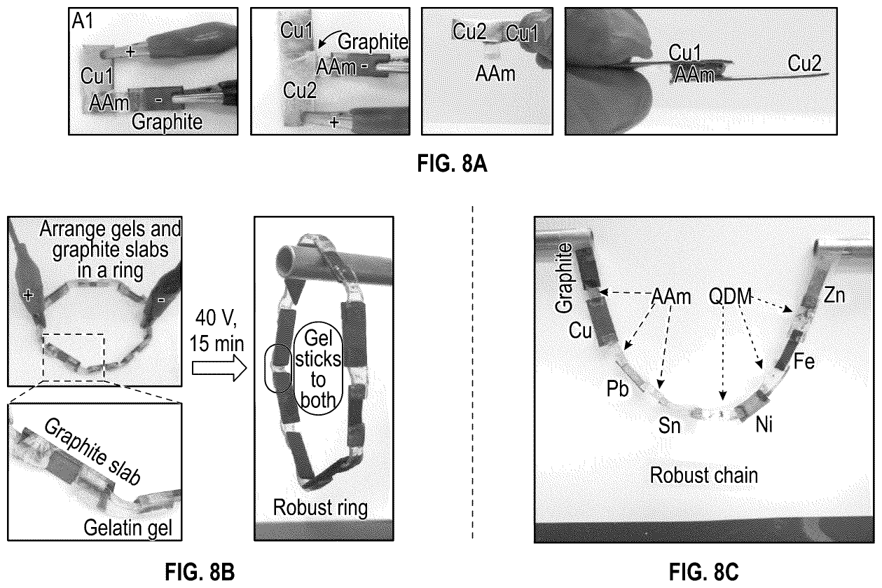

The versatility of EA^{[HS]} can be further shown by sticking hard and soft materials in other geometries or configurations, three of which are shown in FIGS. 8A-8C. First, a thin strip of AAm gel is used as an adhesive to stick two Cu sheets, as shown in FIG. 8A. For this, an AAm gel (20% AAm, with 1% NaCl) is cut into a rectangular (3×1 cm) strip with a thickness of 2 mm. The Cu strips are also similarly sized (but thinner), as shown in the left-side panel of FIG. 8A. Recalling FIG. 5, Cu strips adhere to AAm gels at the anode, whereas neither Cu nor graphite stick to AAm at the cathode, and so a Cu strip is first placed perpendicular to the AAm gel at one end and make this strip the anode (+), as shown in the far left-side panel of FIG. 8A. A graphite slab is placed at the other end of the gel, and made to be the cathode (−). Five volts (5 V) is applied for five minutes (5 min), inducing EA^{[HS]} between the Cu anode and the gel. Then, a second Cu strip is placed over the gel (on the opposite side, parallel to the first Cu strip) and is made the anode, as shown in the middle left-side panel of FIG. 8A. EA^{[HS]} between the second Cu and the gel is then induced, as shown in the middle right-side panel of FIG. 8A. The overhanging portion of the gel is cut off and the two Cu strips stuck together by the AAm gel that is sandwiched between them, as shown in the far right-side panel of FIG. 8A.

Another interesting demonstration is done with gelatin gels and graphite, as shown in FIG. 8B. As noted earlier, gelatin gels adhere to graphite both at the anode and the cathode. A single-step adhesion of gelatin and graphite pieces into a closed ring was attempted. For this, eight graphite slabs (3×1×0.2 cm size) and eight gelatin gel strips (3×1×0.2 cm size) were arranged in a ring using Parafilm, as shown in the upper lefthand panel of FIG. 8B. Note that a portion of a given gel strip bridges adjacent graphite slabs, as shown in the lower lefthand panel of FIG. 8B. This is essentially a series configuration of hard and soft materials. Positive and negative terminals are connected to two ends of the ring and apply a direct current (DC) voltage of forty volts (40 V) for fifteen minutes (15 min). Each gel strip has one graphite slab connected to it as the anode and another as the cathode. Due to EA^{[HS]}, all graphite-gel pairs adhere strongly, and the result is a robust ring. The right-side panel of FIG. 8B shows the ring being lifted up in the air by a metal tube. The ring can thus be manipulated as a single object. The ring stays intact without loss of adhesion indefinitely, as long as the gels remain hydrated.

### Mechanism for EA^{[HS]}

The mechanism, i.e., why EA^{[HS]} occurs, is conceptually distinct from all previous uses of the term ‘electroadhesion’. It arises between a hard electronic conductor and a soft, ionic conductor. Once induced by the DC electric field, the adhesion persists thereafter. Adhesion is induced with both chemical and physical gels, as shown in FIGS. 6A-6C. Moreover, adhesion can be achieved to gels that are cationic, anionic, or nonionic. Some anionic gels like SA stick to graphite only at the anode, as shown in FIG. 6A, whereas other anionic gels like alginate stick to graphite only at the cathode FIG. 6B. Thus, electrostatic or ionic interactions cannot be a decisive factor in the mechanism behind EA^{[HS]}.

The results on EA^{[HS]} to AAm gels with different metals follow a significant trend, as shown in FIG. 5. Metals adhere only at the anode to these gels, and those that do have positive reduction potentials, while those that do not have negative reduction potentials. This correlation with the electrochemical series strongly indicates that adhesion is caused by electrochemical reactions between the metal and the gel at the anode. Metals that do not adhere are those that get oxidized first at the anode. This oxidation (electrolysis) of the metal dominates over any competing processes, which explains why there is no adhesion. Conversely, metals that do adhere are relatively inert, allowing the polymer chains of the gel network to get oxidized first at the anode. Such oxidations result in chemical bonds between the metal surface and the polymer chains, leading to adhesion. The precise nature of the bonds will vary depending on the chemistry of the gel. When the polarity is inverted (i.e., the adhered surface is now made the cathode), the reactions at the electrode are reductive, which serves to undo the bonds between the metal surface and the polymer chains. As a result, the gel can now be detached from the metal.

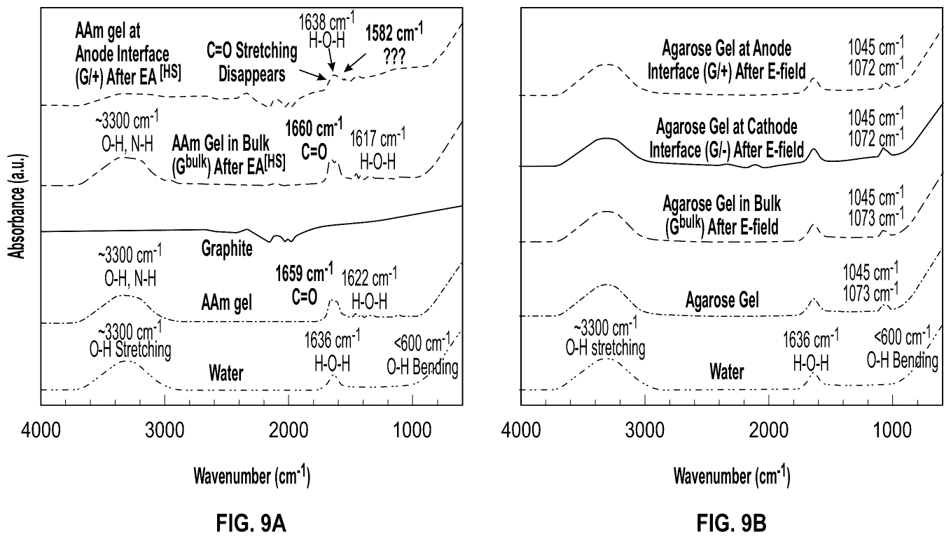

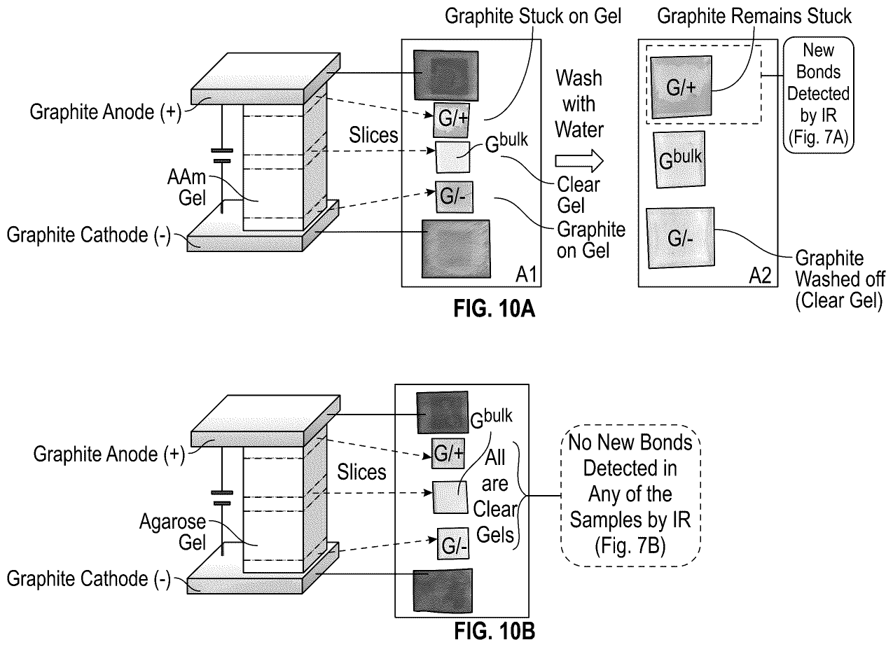

Fourier Transform Infrared spectroscopy (FTIR) in the attenuated total reflectance (ATR) mode was conducted on the graphite-AAm pair, as shown in FIG. 9A. The IR spectrum for a bulk AAm gel is dominated by the water present in it, as can be seen from the bottom two curves. Water shows a broad peak at 3300 cm⁻¹ for O—H stretching, one peak at 1636 cm⁻¹ for H—O—H scissoring, and one below 600 cm⁻¹ for O'H bending. For the AAm gel, all these peaks appear, and there is an additional strong peak at 1659 cm⁻¹ for the stretching of the C═O bond in the amide group. For a polished graphite surface without any contact with gels, IR absorption occurs over the range of wavelengths, but with no clear peaks. Next, graphite electrodes were contacted with the AAm gel and five volts direct current (5 V DC) was applied for fifteen minutes (15 min). As expected, the graphite anode adhered to the AAm gel by EA^{[HS]} while the cathode did not. A razor blade was used to cut slices of the gel next to each electrode as well as in the bulk (middle), i.e., far from the electrode interfaces. Photos of these slices are shown in FIG. 10A and are labeled G/+, G/−, and G^{bulk}.

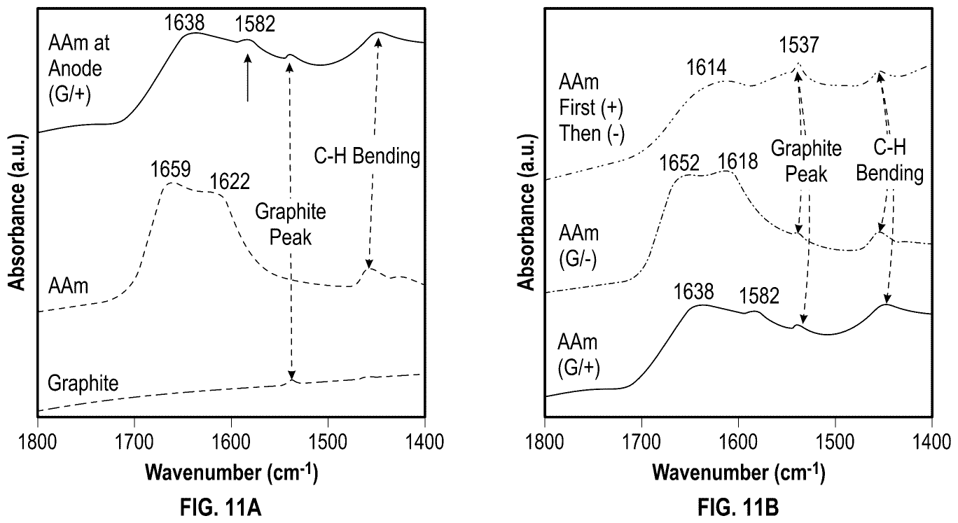

FIG. 9A shows that the AAm gel slice from the bulk (G^{bulk}) has nearly the same infrared (IR) spectrum as the one before adhesion. This indicates that any changes to the gel happen only at the interfaces with the electrodes. Next, the gel slice next to the anode (G/+): from FIG. 10A still has some graphite attached to it and this graphite cannot be washed off with water. The IR spectrum for G/+ is the top (purple) curve in FIG. 9A and it is an overlap of the spectra for graphite and the gel. More importantly, the C═O stretching peak of the amide group has disappeared, while one additional peak now appears at 1582 cm⁻¹. A close-up of this data showing the peaks is provided in FIG. 11A. These data indicate that there must have been electrochemical reactions induced by the field that consume the amide group. In turn, these reactions appear to generate new bonds that are hard to identify. Conversely, the slice of gel next to the cathode (G/−, where there is no adhesion) shows an IR spectrum more similar to the bulk gel, as shown in FIG. 11B. Incidentally, the photos in FIG. 10A show this gel to also have some black graphite on it, but this graphite can be washed off easily, leaving a clear gel.

FTIR was conducted on the graphite-agarose pair, as shown in FIG. 9B. This is a combination for which EA^{[HS]} does not occur. The initial agarose gel has the water peaks as well as additional minor peaks at 1045 and 1073 cm⁻¹. Next, the agarose gel was contacted with graphite and five volts direct current (5 V DC) was applied for fifteen minutes (15 min). A razor blade was then used to cut slices of the gel from the bulk (middle), the anode interface and the cathode interface, as shown in FIG. 10B. Because there is no adhesion, the gel slices at the interfaces have no graphite clinging to them. IR spectra of the three gel slices (top three curves in FIG. 10B are nearly identical and show no new peaks. This indicates that no electrochemical reactions have occurred at the interfaces, or at least none that alter the chemistry of the gel.

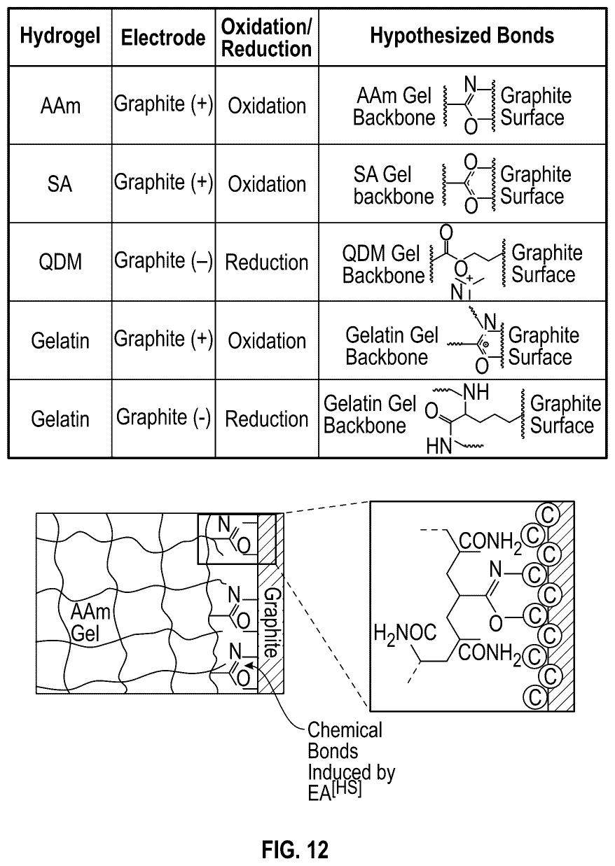

There is much contrast between FIG. 9A and FIG. 9B. For graphite-AAm, where EA^{[HS]} occurs, IR shows evidence of chemical changes to the gel at the adhering electrode after the field is applied. For graphite-agarose, where no such adhesion occurs, there is no evidence of any chemical changes. This strengthens the hypothesis that adhesion arises due to electrochemical reactions between the adhering electrode and the gel. The precise nature of the bonds between the electrode and gel will depend on their chemistries. In the case of the new peak at 1582 cm⁻¹ for AAm-graphite, it cannot be precisely identified from IR databases as to what this peak represents. However, this region of the IR spectrum seems to correspond to alkenes or aromatic rings. As shown in FIG. 12, some possibilities for the bonds that may arise between graphite and AAm as well as a few other hard-soft pairs.

### Applications

A few demonstrations leverage the use of EA^{[HS]}. First, FIGS. 13A-13E show an electro-gripper to pick up and drop off gels. A graphite slab was stuck to the end of a glass rod using epoxy glue and used this graphite as the working electrode (WE). Two pieces of aluminum (Al) foil serve as the counter electrodes. Two DC power sources are used for adhesion and detachment, respectively. The gel cylinder (˜5 cm tall) is made with 20% AAm gel and contains 15% NaCl. The high salt ensures a high ionic conductivity and thereby a short adhesion time. Initially, the gel is placed on the first aluminum foil, as shown in FIG. 13A. The graphite working electrode is placed in contact with the gel and five volts (5 V) is applied for five seconds (5 s), as shown in FIG. 13B: note that the graphite is the anode (+) while the aluminum is the cathode (−). Even with this short time, the graphite strongly adheres to the gel by EA^{[HS]}, allowing the two to be lifted up in the air, as shown in FIG. 13C. The gel-graphite pair is then placed on the second Al foil and a reverse voltage of five volts (5 V) is applied for fifteen seconds (15 s), as shown in FIG. 13D, with the graphite as the cathode (−) and the Al as the anode (+). In this time, the graphite detaches from the gel and can be lifted off, leaving the gel on the Al foil, as shown in FIG. 13E. In this way, the gel is picked up from one spot and dropped off at another. This setup could provide a simpler alternative for grippers in robotics, as it does not require the robot's fingers or hands to have any joints or specific shapes to hold an object.

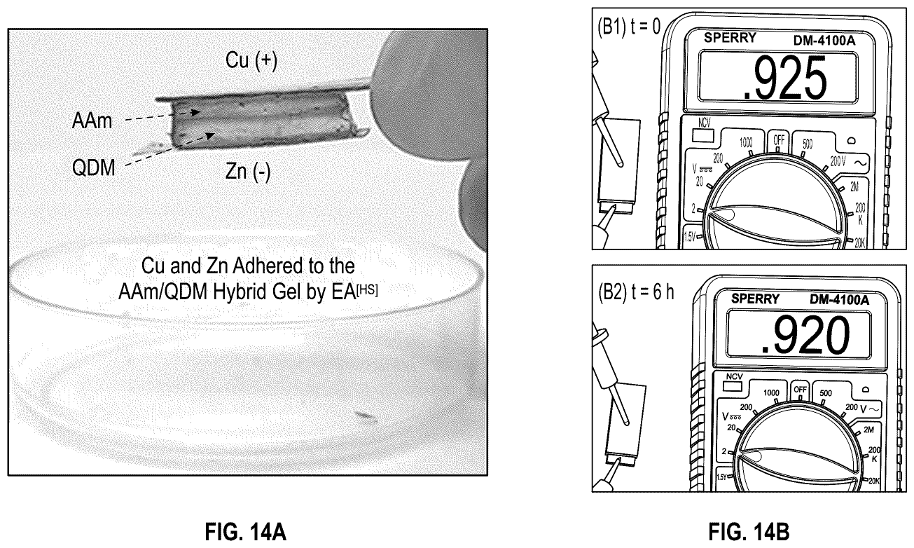

Another potential application of EA^{[HS]} is in making new kinds of batteries. Battery designs often have two hard solids (as electrodes) and an electrolyte between them. The electrolyte can be a soft solid, such as a gel in an ion-conductive solvent. A primary battery was assembled with a hydrogel electrolyte using the EA^{[HS]} technique, as shown in FIG. 14A. The gel is a hybrid composed of two layers, with the top layer being AAm and the bottom being QDM. This hybrid gel was made using a strategy modified from the Experimental section of a previous study. See Banik et al., “A new approach for creating polymer hydrogels with regions of distinct chemical, mechanical, and optical properties” Macromolecules 2012, 45, 5712-5717, which is hereby incorporated by reference herein in its entirety. Cu and Zn were chosen as the electrodes. The logic behind these choices is that AAm adheres to Cu anodes by EA^{[HS]}, as shown in FIG. 5 and FIG. 8A, while QDM adheres to Zn cathodes by EA^{[HS]}, as shown in FIG. 8C. A Cu strip was placed in contact with the AAm side and a Zn strip with the QDM side. The gels both had 1% NaCl in them, as usual, for ionic conductivity. Then, with Cu as the anode and Zn as the cathode, ten volts (10 V) was applied for thirty seconds (30 s). Both the metals adhered to the gels, as shown in FIG. 14A. During this process, Cu was electrolyzed into Cu²⁺ within the AAm gel, and the gel turned blue as a result. The overall assembly serves as a primary battery, as shown in FIG. 14B. The open-circuit potential, with Cu as the cathode (+) and Zn as the anode (−), is ˜0.9 V. This output was stable for hours, as shown in FIG. 14B, which evidences that the setup provides the basic function of a battery. More sophisticated battery designs, including flexible and rechargeable batteries, can be assembled in the future using EA^{[HS]}.

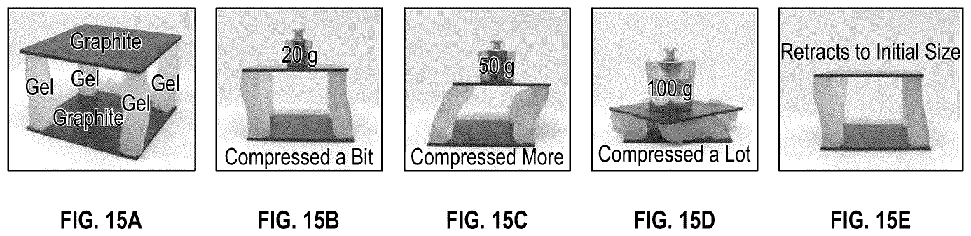

A third potential application of the EA^{[HS]} technique is in bio-inspired actuators and soft robotics. By combining flexible hydrogels with rigid solid materials, robots can be fabricated robots with a stiff, bone-like skeleton as well as soft, muscle-like elements. FIGS. 15A-15E shows a simple example of a load-bearing hard-soft structure made using EA^{[HS]}. Two graphite slabs (5×5 cm) are joined by EA^{[HS]} to four pillars made of flexible AAm gels. To make the gels flexible, the graphite slabs are crosslinked by nanoparticles of the synthetic clay laponite, instead of the molecular crosslinker BIS. Each gel is made in the shape of a long cuboid (0.5×0.5×3 cm) and the gels are all robust and elastic. The gels are fixed as pillars on the four corners between the two slabs, as shown in FIG. 15A. Weights were then placed on the top slab to examine the ability of the structure to support a load. The structure is negligibly affected when twenty grams (20 g) is placed, as shown in FIG. 15B, whereas fifty grams (50 g) makes the pillars buckle and bend, as shown in FIG. 15C. When the load is increased to one hundred grams (100 g), as shown in FIG. 15D, the gel pillars buckle and the top slab is pushed down to the bottom one. When this weight is removed, the gel pillars retract to their original state and so the top slab moves upward, as shown in FIG. 15E.

The results in FIGS. 15A-15E show the utility of combining hard and soft elements in the same structure. Hard elements alone will not be compressible or deformable, whereas soft elements alone could get crushed by a load. The combination, however, is able to bear a load without damage. Moreover, as the pillars retract after load removal, as shown in FIG. 15E, the elastic energy stored in the deformed gels gets released, and in the process, the structure can do work (e.g., push an object). During all these steps, the strong adhesion induced by EA^{[HS]} between the hard slabs and the soft pillars persists and endures. This demonstrates that EA^{[HS]} can indeed be leveraged in making actuators and robots. Similar hard-soft assemblies could also be useful in the body where metal implants like stainless steel or titanium are widely used. The ability to adhere a gel (or tissue) to metal could be useful in reinforcing these implants and also to control the interface between the implant and bodily fluids.

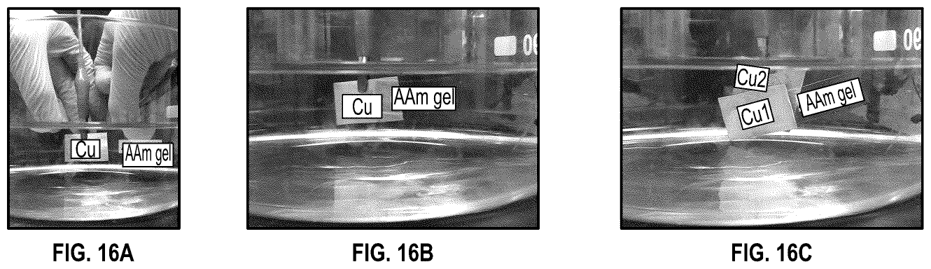

A final point is that the adhesion induced by EA^{[HS]} between a hard and a soft solid can also be achieved underwater. This is demonstrated in FIGS. 16A-16C using Cu sheets and an AAm gel. First, in FIG. 16A, a Cu sheet is brought into contact with a strip of AAm gel while being immersed in water. Cu is made to be the anode (+) and the circuit is completed with graphite as the cathode (−), similar to the arrangement in FIG. 8A. The gel is the same as in FIGS. 13A-13E and has high salt content (15% NaCl), which ensures a short adhesion time. Five volts (5 V) is applied for sixty seconds (60 s), thus inducing EA^{[HS]} between the metal and the gel, as shown in FIG. 16B. Note that the graphite as cathode does not adhere to the gel. Next, a second Cu sheet is stuck to the opposite side of the AAm gel strip. For this, the second Cu sheet is made the anode, graphite is again the cathode, and the adhered Cu sheet is left in open circuit. Five volts (5 V) is again applied for sixty seconds (60 s) to induce EA^{[HS]}. The result, as shown in FIG. 16C, is that the two Cu sheets are stuck together by the AAm gel, similar to the earlier result in FIG. 8A. However, in the present case, the entire assembly is underwater—thus the gel is able to serve as an underwater adhesive.

Achieving adhesion underwater has proven to be a huge challenge in recent years because many flowable adhesives cannot be spread onto solid surfaces that are immersed in liquids like water. Even if spreading can be achieved, the solid-solid adhesion ends up being quite weak because the bonds between the solids are influenced by the water molecules around them. Here, the technology of the present disclosure surmounts this problem because the gel is not inherently adhesive to the metal. The adhesion is only switched on when the gel and metal are contacted under the field.

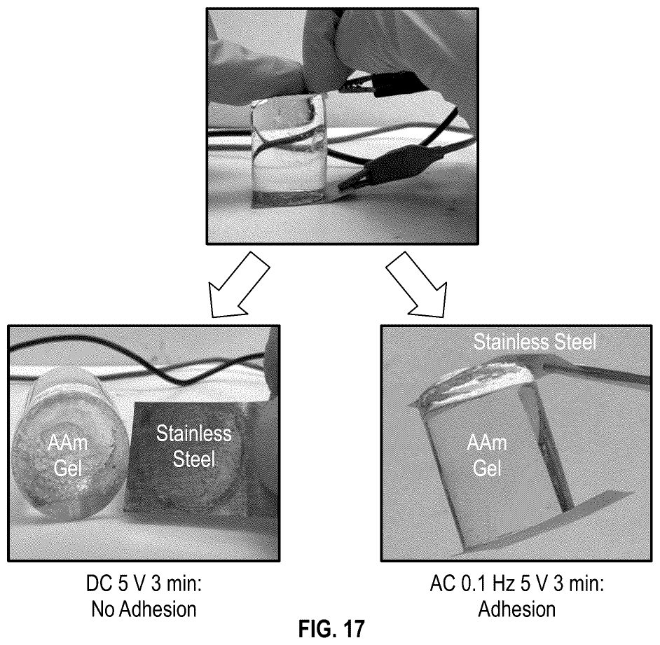

In some cases, alternating current (AC) enables adhesion (EA^{[HS]}) of a metal to a gel while direct current (DC) does not. One specific example, as shown in FIG. 17, shows adhesion of stainless steel (SS), including household objects like a spoon or fork, to a gel of acrylamide (AAm). Adhesion of stainless steel (SS) does not work by applying five volts direct current (5 V DC) for three to five minutes (3 to 5 min), as shown in the lower lefthand panel. Adhesion of stainless steel (SS) does work by applying five volts alternating current (5 V AC) at a frequency of 0.1 Hz for three to five minutes (3 to 5 min), as shown in the lower righthand panel. AC frequency is also important. Only low frequencies, e.g., 0.1 to 0.5 Hz, seem to work.

A systematic set of data shows adhesion (EA^{[HS]}) to tissues of mammals, e.g., pig, cow, chicken. Several conductive hard solids, e.g., graphite, pure metals, and alloys such as stainless steel and nitinol, can be adhered to a variety of tissues by applying five volts direct current (5 V DC) for fifteen minutes (15 min). Table 2 below is for tissues from the pig.

TABLE 2
Adhesion of hard materials to tissues

| | DC, 5 V 15 min | | | | |
|---|---|---|---|---|---|
| | Skin | Brain | Heart | Aorta | Kidney |
| Graphite | + and − | + and − | + and − | + and − | + and − |
| Stainless | + | + | + | + | + |
| Steel | | | | | |
| Copper | + | + | + | + | + |
| Silver | No | No | + | + | No |
| Nitinol | | | | + | |

With many metals, adhesion of the tissue is only to the + electrode. However, in the case of graphite, the tissues adhere at both the + and − electrodes.

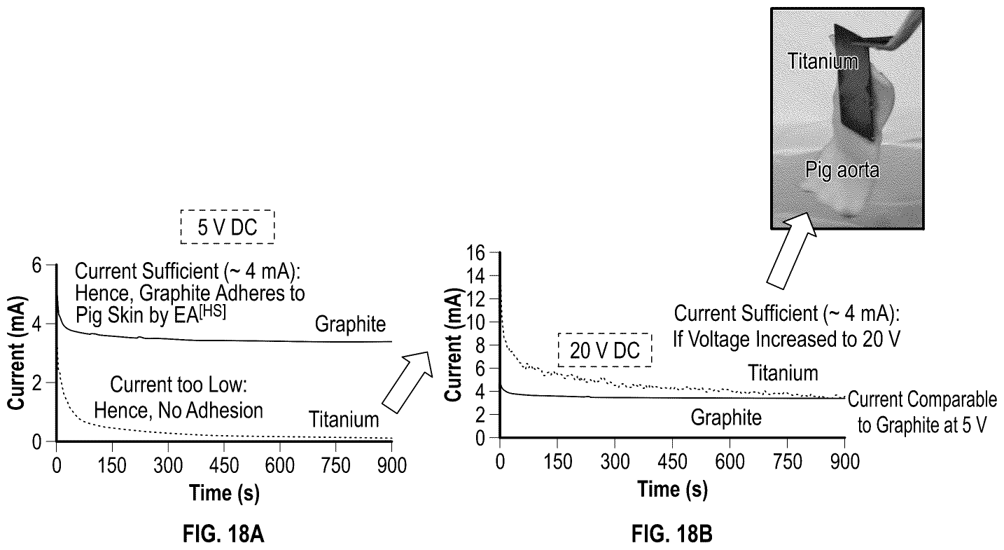

In some cases, if adhesion by direct current (DC) of a metal to a gel or soft material is not observed, the reason could be a low current. If so, increasing the voltage (and thereby the current) can enable adhesion (EA^{[HS]}). One specific example, as shown in FIGS. 18A-18B, shows adhesion to metals, e.g., titanium, to a tissue, e.g., a pig aorta. As shown in FIG. 18A, the adhesion does not work by applying five volts direct current (5 V DC) for fifteen minutes (15 min). The current is too low. As shown in FIG. 18A, the adhesion does work by applying twenty volts direct current (20 V DC) for fifteen minutes (15 min). The current is much higher.

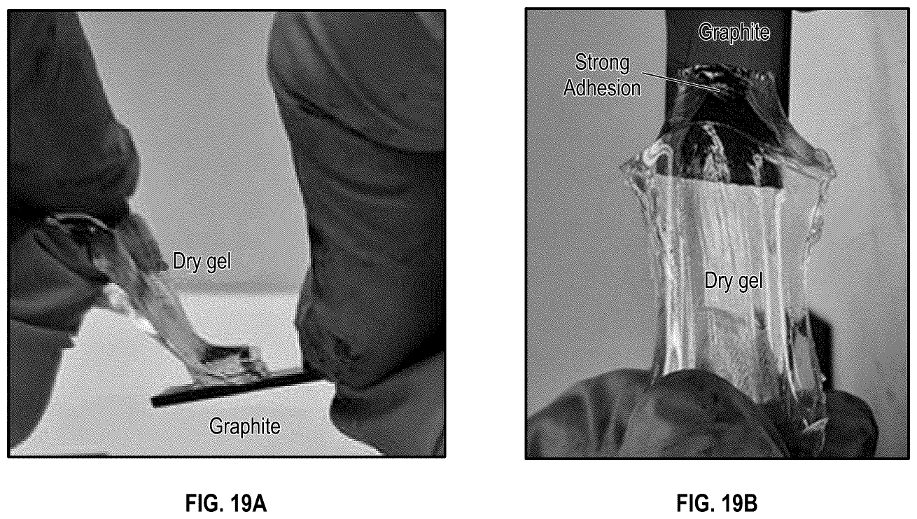

EA^{[HS]} can be used to adhere graphite and metals to soft materials that are dry, e.g., no water present, provided the soft material is ionically conductive. Likewise, soft gels that contain no water can be adhered to, but instead contain a non-aqueous polar liquid, e.g., an organic solvent, provided that the gels are ionically conductive. One specific example, as shown in FIGS. 19A-19B, shows adhesion of graphite to a dry gel of gelatin (which could comprise an organogel). Gelatin gel (10% gelatin) with 0.9% NaCl and 20% glycerol was prepared, then dried overnight. Glycerol keeps the gel flexible because the glycerol is a plasticizer. NaCl (salt) ensures ionic conductivity. Graphite is adhered to the above dry gel by applying fifteen volts (15 V) for three minutes (3 min).

### Conclusion

In summary, a method for adhering hard materials to soft, aqueous materials is disclosed herein. The hard material is an electronic conductor like a metal or graphite, which allows the hard material to serve as electrodes in a simple electrochemical setup. The soft material is an ionic conductor, which typically means it must have water and ions (salt). Examples of such materials include hydrogels as well as plant-based tissues (fruits and vegetables) and animal-based tissues (meat from cows, pigs, and chickens). The method is termed “hard-soft electroadhesion” or EA^{[HS]}. The method brings the hard and soft material into contact and apply a low DC electric field (e.g., 5 V) for a short time (e.g., 3 min). Depending on the nature of the hard and soft materials, adhesion is induced at the anode (+), cathode (−), both electrodes, or neither. This adhesion endures after the field is removed. If adhesion is observed only to one electrode, switching the polarity of the field typically reverses the adhesion. The adhesion strength increases with increasing voltage, time in the field, and ionic conductivity of the gel. The ultimate adhesion strength is limited only by the strength of the gel. Metals that can be adhered via EA^{[HS]} to AAm gels (all at the anode) have reduction potentials above a critical value. This correlation to the electrochemical series suggests that EA^{[HS]} is due to chemical bonds between the gel and the anode induced by electrochemical reactions. Support for this conclusion comes from FTIR data. Finally, the versatility of this phenomenon is shown through various examples. EA^{[HS]} could enable applications in robotics, energy storage, and biomedical implants.

### EXPERIMENTAL SECTION

### Materials

The following monomers were from Sigma-Aldrich: acrylamide (AAm), N,N-dimethylacrylamide (DMAA), N-isopropyl-acrylamide (NIPA), 2-hydroxyethyl methacrylate (HEMA), 2-(di-methylamino)ethyl methacrylate (DMAEMA), sodium acrylate (SA), [2-(methacryloyloxy)ethyl]trimethyl-ammonium chloride (QDM, 75% solution in H₂O), and N,N′-methylene-bis (acrylamide) (BIS). Other chemicals used for making hydrogels were also from Sigma-Aldrich, including the initiators ammonium persulfate (APS) and potassium persulfate (KPS) and the accelerant N,N,N′,N′-tetra-methylethylenediamine (TEMED). Polymers used in this study were also from Sigma-Aldrich and included alginate (i.e., alginic acid sodium salt, from brown algae, medium viscosity), agarose (Type IA, low EEO), gelatin (from porcine skin, gel strength 300, Type A), and poly (vinyl alcohol) (PVA, MW 85-124k, 99+% hydrolyzed). Other chemicals included calcium chloride dihydrate (CaCl₂, from Sigma-Aldrich), sodium chloride (NaCl, from LabChem) and hydrochloric acid (HCl, from BDH). Graphite sheets (˜1.5 mm thickness) were from Saturn Industries. Dyes used to color-code the gels were methyl orange from TCI America, and methylene blue and rhodamine B from Sigma-Aldrich. Laponite XLG was a gift from Southern Clay Products. Cu, Pb, Sn, Ni, Fe, Zn and Ti were purchased from RotoMetals. All the meat, fruits and vegetables were purchased from Whole Foods. Deionized (DI) water was used in all experiments.

### Hydrogel Synthesis

AAm, SA, DMAA, NIPA, QDM, DMAEMA and HEMA gels were prepared by free-radical polymerization. For a typical gel, 20% monomer, 0.03-0.06% BIS (crosslinker), and 2.0 μL/g TEMED (accelerator) were dissolved in DI water. After sufficient mixing, 0.02-0.06% initiator (APS or KPS) was quickly mixed into the solution. Then the solution was placed under a nitrogen atmosphere for at least 30 min, whereupon it was polymerized into a gel. For laponite-crosslinked AAm gels, 3% laponite XLG particles were used as crosslinker instead of BIS. Gels were typically made in 30 mL cylindrical vials and when taken out of the vial, they had a cylindrical form with a size close to the vial dimensions, i.e., a height of five centimeters (5 cm) and a diameter of 2 cm. Such cylindrical gels were used in the adhesion studies shown in FIGS. 1A-1B, FIG. 5, and FIGS. 6A-6C. For other experiments, the same gels were also made in Petri dishes. For all electroadhesion experiments, it was necessary to include salt in the gel to ensure ionic conductivity. For this purpose, 1% NaCl was typically added to the monomer solution prior to polymerization.

Alginate gels were made by a new method that involves diffusion of Ca²⁺ cations. 3% alginate was dissolved in DI water in a vial. Then the solution was frozen at −20° C. overnight. The vial was then broken and the frozen solid was removed. This cylindrical solid (˜2 cm diameter and 5 cm height) was then placed in a 7% CaCl₂ solution at room temperature on a stir plate. As the solid melted, Ca²⁺ came into contact at the interface and the cations diffused inwards to crosslink the alginate chains. Within 6 h, an alginate gel in the shape of a cylinder was obtained, and this was used in FIGS. 6A-6C.

PVA gels were made by freeze-thaw cycling. 20% PVA (with 1% NaCl for ionic conductivity) was dissolved in DI water at ˜90° C. Upon cooling to room temperature, a viscous solution was obtained. This solution was then subjected to two freeze-thaw cycles. For each cycle, the solution was frozen at −20° C. overnight and then thawed at room temperature. The freeze-thaw cycling induced the PVA to form a robust gel due to the formation of crystallites at junctions between the chains.

Gelatin gels were made by dissolving 20% gelatin together with 1% NaCl in DI water at 50° C. Upon cooling to room temperature, the solution was converted into a gel due to the formation of triple-helical junctions between the gelatin chains. Similarly, agarose gels were made by heating 5% agarose together with 1% NaCl in DI water at 90° C. Upon cooling to room temperature, the agarose chains bind to each other via hydrogen-bonds, resulting in a gel.

The hybrid AAm/QDM gel was made using a method modified from a previous work. See Banik et al., “A new approach for creating polymer hydrogels with regions of distinct chemical, mechanical, and optical properties” Macromolecules 2012, 45, 5712-5717, which is hereby incorporated by reference herein in its entirety The QDM gel was first made in a 90×10 mm Petri dish, as described above. After the QDM gelled, a pre-gel solution of AAm was added onto the top. Then the sample was placed again under a nitrogen atmosphere until the AAm also gelled. The AAm and QDM layers were strongly bonded with each other since the pre-gel solution would diffuse into the QDM gel, making the resulting AAm gel network penetrate with the existing QDM network. Then the hybrid gel was cut into appropriate shape with a razor blade for adhesion experiments.

### Electroadhesion Experiments

Two polished slabs or strips of the hard material (graphite or metal) were placed in contact on either end of the gel (or other soft material) to be tested, as shown in FIGS. 1A-1B. The two slabs were connected as electrodes to the positive and negative terminals of a BK Precision 9104 DC power supply. A DC voltage of typically five to ten volts (5 to 10 V) was applied for three to fifteen minutes (3 to 15 min). If the duration of exposure to the electric field was relatively long, the gels were covered with Parafilm during the experiment to minimize water evaporation. After stopping the field, the electrodes were examined for adhesion to the gel. Next, the polarity was reversed (i.e., the previous anode became the cathode, and vice versa) and the field was reapplied. The electrodes were then examined again for adhesion to the gel.

### Pull-Off Adhesion Strength Tests

The data shown in FIGS. 2A-2F are for the strength of EA^{[HS]} between AAm gels and graphite. For these measurements, AAm gels were made in Petri dishes, from which they were cut into cuboids with a square cross-section (1×1 cm) and a height of 1 to 1.6 cm. The gel was then electroadhered to a graphite slab (2×1.5×0.2 cm). After adhesion, the gel was cut open using a razor blade with ˜0.2 cm thickness left on the graphite slab. This gel-graphite pair was then taken for pull-off testing on an AR2000 rheometer (TA Instruments) using 40-mm parallel-plates. As shown in FIG. 3A, the back side of the graphite was stuck to the bottom plate using either double-sided tape or an epoxy glue. On the top plate, a zinc sheet was first affixed using double-sided tape or epoxy glue. Then, the gel was stuck to the sheet using a cyanoacrylate glue. With this setup, the pull-off test mode was selected on the rheometer software. During the test, the top plate was pulled upward at a constant rate (typically 1.0-9.3 μm/s; the stiffer the gel, the lower the rate) and the normal-force transducer was used to record the normal force as a function of the gap (distance). The force was converted to stress by dividing by the contact area between the gel and the graphite. The stress at the point of failure was taken as the pull-off strength for a given gel-graphite pair.

### Rheology

Rheological experiments on the AAm gels (FIG. 4A-4B) were performed at 25° C. on an AR2000 stress-controlled rheometer (TA Instruments) using twenty millimeter (20 mm) parallel plates. Gels were cut into discs of a diameter of twenty millimeters (20 mm) and a thickness of two millimeters (2 mm). Dynamic stress-sweeps were first performed to identify the linear viscoelastic (LVE) region of the sample. Dynamic frequency sweeps were then conducted at a constant strain amplitude within the LVE region.

### Infrared Spectra

Infrared spectra were collected using a Fourier transform infrared (FTIR) spectrometer (Thermo Nicolet NEXUS 670) in attenuated total reflectance (ATR) mode. Samples examined were typically those at gel-electrode interfaces (FIGS. 10A-10B). Samples were directly placed on the ATR window in the instrument and absorption spectra were measured over a wavenumber range of 650-4000 cm⁻¹ with a resolution of 4 cm⁻¹. Each sample underwent 32 scans, and the resulting spectra was averaged.

### Statistics

For the data in FIGS. 2A-2F, at least three samples were tested for each data point. No outliers were excluded. Mean values are shown in the plots and error bars correspond to standard deviations. Statistics were calculated and plotted using Excel and SigmaPlot.

From the foregoing, it can be seen that the present disclosure accomplishes at least all of the stated objectives.

### GLOSSARY

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order (e.g., first, second, third), a position, and/or an orientation are not limiting and are only referenced according to the views presented.

When an element is referred to as being “connected,” “coupled,” “mated,” “attached,” “fixed,” etc. to another element, the element can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.). Similarly, terms such as (i) “communicatively connected” or (ii) “fluidly connected” include (i) all variations of information exchange and routing between two electronic devices, including intermediary devices, networks, etc., connected wirelessly or not and (ii) all variations of fluid exchange and routing between two fluidic bodies, including intermediary fluid paths, flows, etc., connected indirectly or not.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A method of hard-soft electroadhesion (EA^{[HS]}), comprising:
conducting electrons with a hard material;
conducting ions with a soft material; and
applying an electric field so as to adhere the hard material and the soft material to one another;
wherein adhesion of the soft material and hard material endures after the electric field is removed.

2. The method of claim 1, further comprising adding salt to the soft material.

3. The method of claim 1, further comprising unsticking the hard material from the soft material by reversing the electric field.

4. The method of claim 1, wherein a strength of the electric field is no less than three volts (3 V) and no more than twenty volts (20 V).

5. The method of claim 1, wherein the electric field is applied for a duration of time between thirty seconds (30 sec) and fifteen minutes (15 min).

6. The method of claim 1, wherein the hard material is selected from the group consisting of: a carbon-based mineral, a metal, and an alloy.

7. The method of claim 6, wherein the hard material comprises titanium, stainless steel, or nitinol.

8. The method of claim 7, wherein the soft material comprises a tissue of an animal.

9. The method of claim 6, wherein the hard material comprises graphite.

10. The method of claim 1, wherein the soft material is a hydrogel that comprises water.

11. The method of claim 10, wherein the soft material comprises a reduction potential greater than 0.

12. The method of claim 10, wherein the soft material comprises a cylindrically shaped form.

13. The method of claim 1, wherein the soft material comprises plant material.

14. The method of claim 1, further comprising correlating pull off strength to one or more of a duration in which the electric field is applied, a strength of the electric field, or a salt concentration in the soft material.

15. The method of claim 1, further comprising selecting to induce adhesion at an anode or a cathode based on a type of the soft material.

16. The method of claim 1, wherein the soft material is capable of adhering to both an anode and a cathode.

17. A reversible system comprising:
a hard material capable of conducting electrons;
a soft material capable of conducting ions; and
a battery that can apply an electric field such that the hard material and the soft material adhere to one another.

18. The reversible system of claim 17, wherein the reversible system is located underwater.

19. The reversible system of claim 17, wherein the reversible system comprises an armor or an implant for a living organism.

20. The reversible system of claim 17, wherein the reversible system comprises a structure formed from at two hard materials adhered to one or more soft materials.
